(19) United States
(12) Patent Application Publication
(10) Pub. No.: US 2008/0221420 A1
(43) Pub. Date: Sep. 11, 2008

(54) Fetal Pulse Oximetry and ECG Sensor

(75) Inventors: Vladimir Grubac, Brooklyn Park, MN (US); Peter R. Rosendahl, Minneapolis, MN (US); Douglas R. Maser, Minneapolis, MN (US); Philip O. Isaacson, Chanhassen, MN (US)

Correspondence Address: FULBRIGHT & JAWORSKI L.L.P., 80 SOUTH EIGHTH STREET, SUITE 2100, MINNEAPOLIS, MN, 55402, US

(73) Assignee: Nonin Medical, Inc., Plymouth, MN (US)

(21) Appl. No.: 11/683,777

(22) Filed: Mar. 8, 2007

(51) Int. Cl. A61B 5/1464

(52) U.S. Cl. 600/338

(57) ABSTRACT

A medical device configured to be temporarily secured at a tissue field, such as a fetal skull, via a spiral probe. The spiral probe functions to both secure the sensor in place and provide an electrode for ECG purposes. The spiral probe is non-uniform and includes one or more of a stop element, a cross bar element and a collapsed portion adapted to engage tissue after a predetermined rotation of the probe into the tissue field. The probe diameter can expand with an increase in torque applied to a drive rod, leading to disengagement of the drive rod from the probe. The spiral probe and drive rod may define a detent mechanism whereby upon reaching a predetermined torque the drive rod is disengaged from the probe and freely rotates without further entry of the spiral probe into the tissue field.

### TECHNICAL FIELD

The present disclosure is directed to medical sensing devices. More specifically, the present disclosure is directed to a sensor device and method of use for measuring vital parameters of a fetus during birth.

### BACKGROUND OF THE INVENTION

Fetal monitoring has been used to prevent injury to the most vital and sensitive organs, such as the brain and the heart, by detecting a decreased oxygen supply to these organs before the onset of cell damage. Some causes of fetal hypoxia are umbilical cord compression, placental insufficiency or hypertonia of the uterus. Early examples of fetal monitoring are intermittent auscultation of fetal heartbeat, electronic monitoring of fetal ECG and heart rate, and scalp blood pH. These techniques are based on the assumption that fetal hypoxia, leads to fetal acidemia and also to specific pathologic fetal ECG and heart rate patterns. These indirect techniques, however, are unsatisfactory because it is only after hypoxia has occurred for some time that it is reflected in adverse changes in the heart rate or blood pH.

Fetal assessment has evolved to the direct measurement of fetal oxygen status using pulse oximetry. Pulse oximetry instrumentation, which provides a real-time measurement of arterial oxygen saturation, has become the standard of care for patient vital sign monitoring during anesthesia and in neonatal and adult critical care. A pulse oximetry system consists of a sensor attached to a patient, a monitor, and a lead connecting the sensor and monitor. The sensor typically has red and infrared light emitting diodes that illuminate a tissue site and a photodetector that measures the intensity of that light after absorption by the pulsatile vascular bed at the tissue site. From these measurements, the oxygen saturation of arterial blood can be calculated.

Pulse oximetry as applied to fetal intrapartum monitoring must overcome several significant and interrelated obstacles not faced by pulse oximetry as applied to adults, children, infants and neonates. These obstacles include attaching the sensor to a readily accessible tissue site, obtaining a representative measurement of central arterial oxygen saturation at that site, and calibrating the sensor. Pulse oximetry sensors are conventionally attached, for example, to an adult finger or a neonate foot using a self-adhesive mechanism that wraps around the tissue site. Sensor attachment to a fetus in this manner is impractical if not impossible. Further, the presenting portion of the fetus is typically the crown of the head, which yields only the fetal scalp as a readily accessible tissue site. A number of mechanisms have been developed to overcome these impediments to attachment of a pulse oximetry sensor to the fetus. These include suction cups, spiral clamps and vacuum devices for scalp attachment. There are also devices that slide beyond the fetus presenting portion, wedging between the uterine wall and the fetus.

U.S. Pat. Nos. 5,529,064; 5,911,690 and 5,865,737, incorporated by reference herein, by Rall and Kintza, disclose a scalp attachment mechanism used in conjunction with a fetal ECG sensor. The sensor assembly consists of a fetal sensor, a driver within a guide tube to facilitate placement, and interconnecting conductors for communication signals to a monitor. The fetal sensor has a spiral probe attached to a sensor base. The probe is utilized to attach the sensor to the fetal scalp and also functions as an ECG probe. The sensor base is removably connected to the driver. The driver is movably contained within the guide tube. The interconnecting wires are attached at one end to the sensor base, and one of the conductors is electrically connected to the probe.

### BRIEF SUMMARY OF THE INVENTION

The present invention is directed to a medical sensor device configured to be temporarily secured at a tissue field, such as a fetal skull, via a spiral probe. The spiral probe functions to both secure the sensor in place and provide an electrode for ECG purposes. The sensor device also includes a housing carrying a light detector and light source utilized during a pulse oximetry process. In some embodiments the spiral probe is non-uniform and includes portions with different diameters and different spiral pitches. In one embodiment, the spiral probe includes a stop element which limits the extent to which a drive rod can be inserted into the probe. In another embodiment, the spiral probe includes a cross bar which engages a portion of the drive rod during placement of the sensor probe. In yet another embodiment, the spiral probe includes a collapsed portion adapted to engage tissue after a predetermined rotation of the probe into the tissue field. The collapsed portion can provide an increased rotational resistance to the drive rod leading to rotational disengagement of the drive rod from the spiral probe. In one embodiment, the spiral probe is directly coupled to an end of the drive rod. The probe diameter can expand with an increase in torque applied to the drive rod, leading to a disengagement of the drive rod from the probe. In one embodiment a spiral probe and drive rod define a detent mechanism whereby upon reaching a predetermined torque the drive rod is disengaged from the probe and rotates without further entry of the probe into the tissue field.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a perspective illustration of one embodiment of a medical sensor system utilizing a sensor device in accordance with the present invention.

FIG. 2 illustrates a partially disassembled sensor device of FIG. 1.

FIG. 3 illustrates a flex circuit aspect of the device of FIG. 1.

FIGS. 4 and 5 illustrate conductive surfaces on the flex circuit of FIG. 3.

FIGS. 6 and 7 illustrate a housing base and collar assembly of the embodiment of FIG. 1.

FIGS. 8 and 9 illustrate top and bottom perspective views of a cover of the embodiment of FIG. 1.

FIGS. 10 and 11 illustrate top and bottom perspective views of a cap of the embodiment of FIG. 1.

FIGS. 12 and 13 illustrate an embodiment of drive rod suitable for use with an embodiment of the present invention.

FIG. 14 illustrates a perspective view of a retractor suitable for use with an embodiment of the present invention.

FIGS. 15 and 16 illustrate perspective views of a spiral probe of FIG. 1.

FIGS. 17-20 illustrate perspective views of a spiral probe and drive rod of FIG. 1.

FIG. 21 illustrates a cross sectional view of a sensor device of FIG. 1.

### DETAILED DESCRIPTION OF THE INVENTION

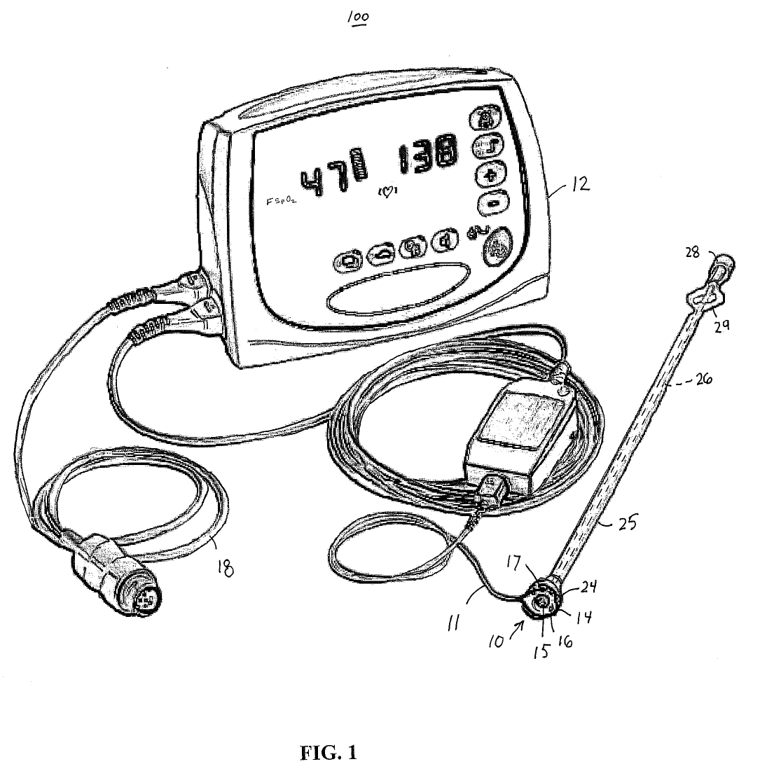

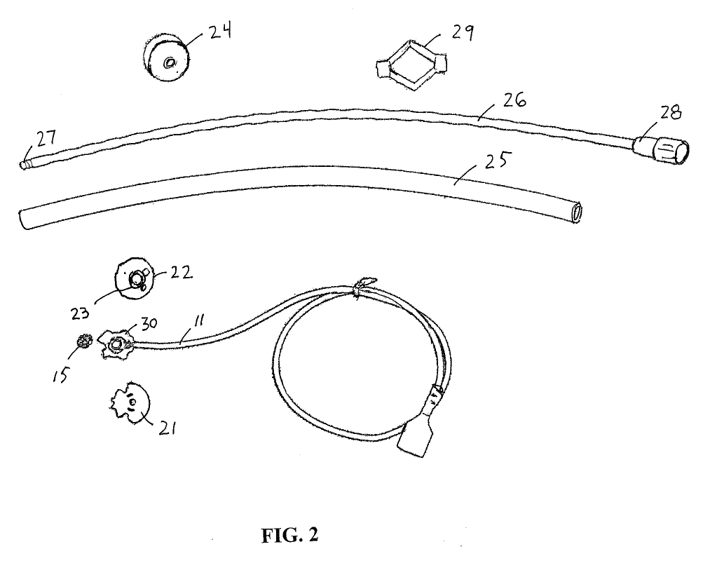

FIGS. 1 and 2 illustrate aspects of a fetal pulse oximetry system 100 in accordance with the present invention. System 100 includes a fetal sensor 10 connected via a communications link, which in this embodiment includes lead 11, to a fetal pulse oximetry/electrocardiogram (ECG) monitor 12. Monitor 12 displays real time fetal pulse oximetry (FsP0₂) and fetal pulse rate (FPR). Fetal sensor 10 attaches in a manner similar to scalp electrodes and passes fetal ECG data to an intrapartum fetal monitor (not shown).

Fetal sensor 10 includes a sensor housing 14 which carries a spiral probe 15, light emitter 16 and light detector 17, which may be a photodiode. Spiral probe 15 is attached to a front end of sensor housing 14 and extends away from the housing 14. Lead 11 is connected at one end to sensor housing 14 and connects to monitor 12 at the other end. Lead 11 transmits signals between monitor 12 and sensor 10. Monitor 12 controls operation of sensor 10 and processes light intensity signals from the light detector 17, providing a display and/or record of the resulting oxygen saturation, pulse rate and plethysmograph. In one embodiment, monitor 12 receives ECG signals from sensor device 10 and provides an interface to a remote fetal ECG monitor, such as via lead 18 (FIG. 1).

Lead 11 is in one embodiment a series of wires that are connected to a remote monitoring device. The remote monitoring device can be in the same room as the patient or can be located elsewhere. However, in some embodiments a wireless communication component may be provided upon or within sensor device 10 to wirelessly communicate to a remote monitor via, for example, one of many known medical device wireless protocols (e.g., BLUETOOTH).

FIG. 2 illustrates an exploded perspective view of components of sensor system 100. Housing 14 includes cover 21 and base 22 which together enclose upon assembly a portion of flex circuit 30, a portion of spiral probe 15 and collar 23. Cap 24 provides a temporary shield around sensor device 10 useful during sensor 10 placement. Cap 24 is secured at one end of guide tube 25 and is removed along with tube 25 subsequent to sensor 10 placement. Electrode drive rod 26 is received within tube 25 and is operatively coupled to rotate spiral probe 15 within sensor 10. Drive rod 26 includes a configured end 27 adapted to engage spiral probe 15 and an opposite end defining handle 28 adapted to be grasped and rotated during sensor 10 placement. A flexible retractor 29 is connected between an end of tube 25 and drive rod 26. Manipulation of retractor 29 during sensor 10 placement causes drive rod 26 to axially disengage sensor 10, as described in more detail herein after. Together, cap 24, guide tube 25, guide rod 26, handle 28 and retractor 29 define an applicator used to position sensor 10 during a placement procedure.

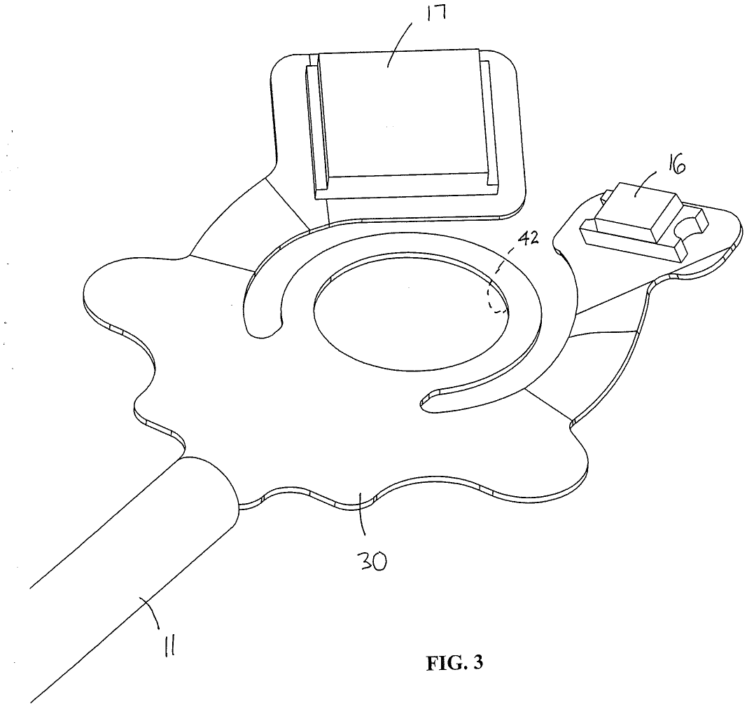

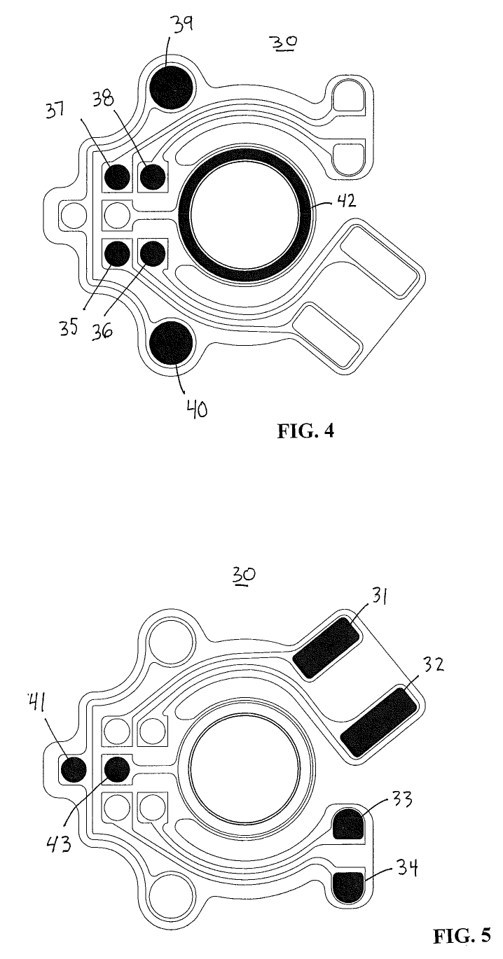

Light detector 17 and light emitter 16 are mounted on a surface of flexible circuit 30. One embodiment of flex circuit 30 is shown in FIGS. 3, 4 and 5. Flex circuit 30 includes pads 31, 32 for surface mounting light detector 17 and pads 33, 34 for mounting light emitter 16. Pads 31, 32 are electrically connected to pads 35, 36, and pads 33, 34 are electrically connected to pads 37, 38 via conductors upon flex circuit 30. Conductive pads 39, 40 are ground pads used to couple sensor 10 to a reference potential, e.g., established by amniotic fluid. Pads 39, 40 are connected via a conductor to pad 41. Conductive ring 42 is electrically connected to pad 43 via a conductor upon flex circuit 30. Lead 11 includes a plurality of wires which are electrically connected to pads 35, 36, 37, 38, 41, 43. During operation of sensor system 100, pads 35 and 36 are within a communications circuit providing a light detector signal from light detector 17 to monitor 12. Pads 37 and 38 are within a power circuit providing power to light emitter 16 from monitor 12. Pads 41 and 43 are within a communications circuit providing an ECG signal to monitor 12.

Detector 17 and emitter 16 are mounted on one side of the flex circuit 30 substrate. Detector 17 and emitter 16 may be partially enclosed in an encapsulant. Detector 17 is mounted so that the active, light collecting region of the photodiode faces the same housing side as spiral probe 15. Emitter 16 contains a pair of light emitting diodes (LEDs), one of which emits a narrow band of red wavelength light and the other of which emits a narrow band of infrared wavelength light. These emitters are mounted so that the active regions of the LEDs face the same housing side as spiral probe 15.

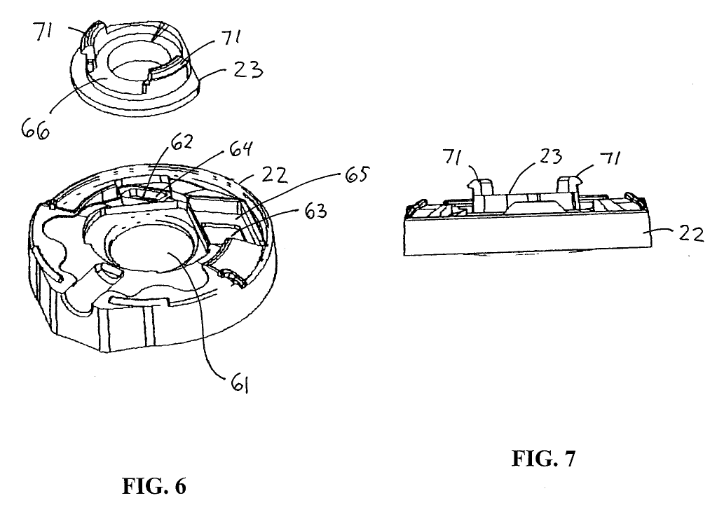

FIGS. 6 and 7 illustrate collar 23 and housing base 22. FIG. 6 shows a disassembled perspective view of the assembly and FIG. 7 shows a side elevational view of the assembly. Base 22 preferably is a soft plastic material which easily conforms to fetal anatomy. A variety of other materials may be practicable for base 22. Base 22 includes a central aperture 61, a light emitter aperture 62 and a light detector aperture 63. Base 22 further defines cavity 64 for receiving a portion of light emitter 16 and cavity 65 for receiving a portion of detector 17. The elasticity of base 22 ensures that it remains in contact with the fetal scalp so that minimal extraneous light can penetrate the base periphery and be detected by light detector 17. Collar 23 is preferably molded into base 22 during a manufacturing process. Collar 23 is relatively rigid and includes a pair of prongs 71 which pass through slots 81 of housing cover 21 to provide a snap fit connection. Collar 23 defines an annular surface 66 which engages a ring portion of flex circuit 30.

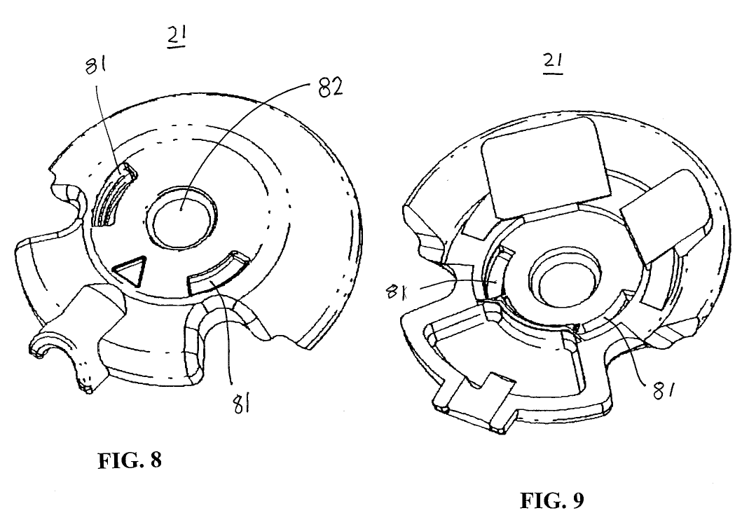

FIGS. 8 and 9 illustrate top and bottom views of housing cover 21. Cover 21 includes a central aperture 82 through which an end portion of spiral probe driver 26 is passed. Cover 21 also includes slots 81 through which prongs 71 of collar 23 pass through during assembly of sensor 10. Cover 21 is preferably formed of a material of compatible hardness as collar 23. Cover 21 and base 22 are secured together at least in part by a mechanical connection including prongs 71 and slots 81. In other embodiments, cover 21 and base 22 may be secured together with an adhesive or another type of mechanical fastener.

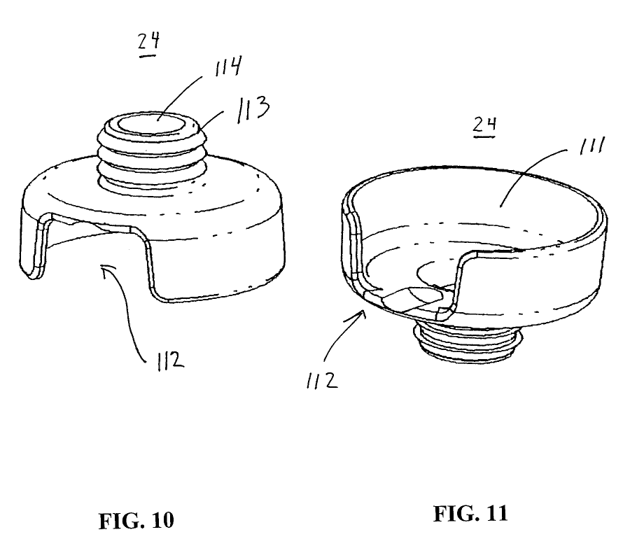

FIGS. 10 and 11 illustrate perspective views of cap 24. Cap 24 defines a generally closed interior 111 for receiving sensor 10. Opening 112 in cap 24 permits lead 11 to pass directly through the cap 24 perimeter. Cap 24 includes end 113 which connects to an end of tube 25. Aperture 114 in cap 24 permits drive rod 26 to pass through to engage sensor 10.

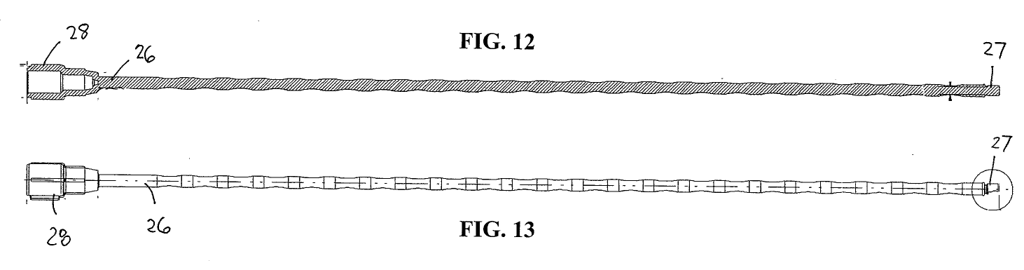

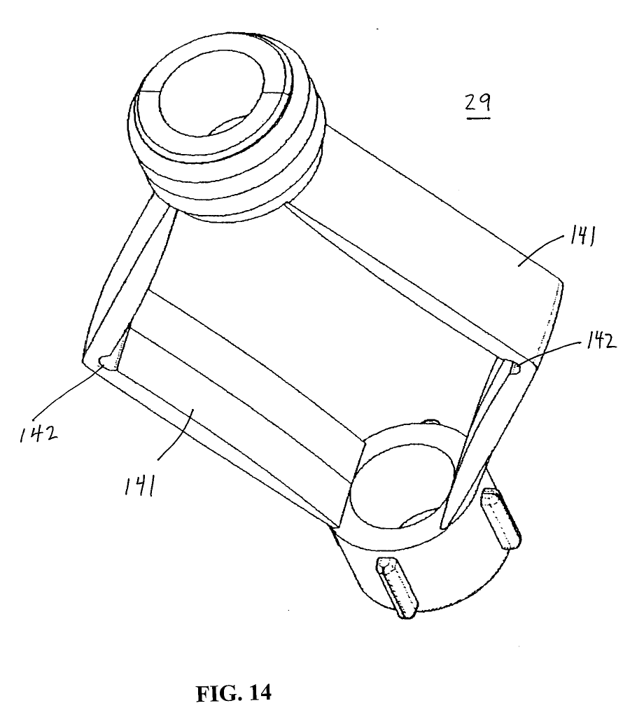

FIGS. 12 and 13 illustrate an embodiment of drive rod 26. Rod 26 is a flexible material adapted to conform to anatomy during placement of sensor 10. Rod 26 is sufficiently rigid to enable a torque transfer throughout its length, e.g., a rotational (torsional) force applied to rod handle end 28 is transferred to end 27 and spiral probe 15. During a placement procedure, a torsional force is applied at handle end 28 by a health care practitioner causing spiral probe 15 to rotate into engagement with a tissue field of, for example, the fetal scalp.

FIG. 14 is an illustration of retractor 29 which is connected between an end of tube 25 and drive rod 26. Retractor 29 includes flexible arms 141 defined by a pair of flexible “living” hinges 142. Inward compression of flexible arms 141 during sensor 10 placement causes drive rod 26 to axially disengage from sensor 10.

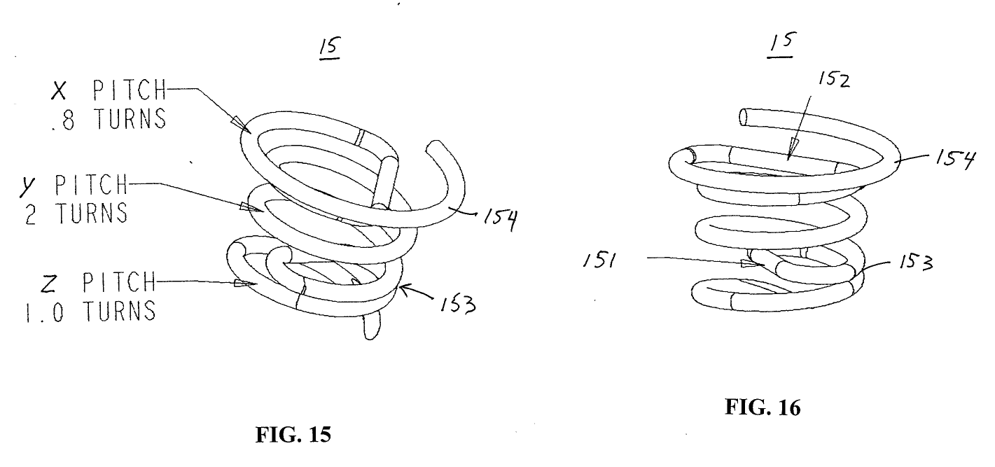

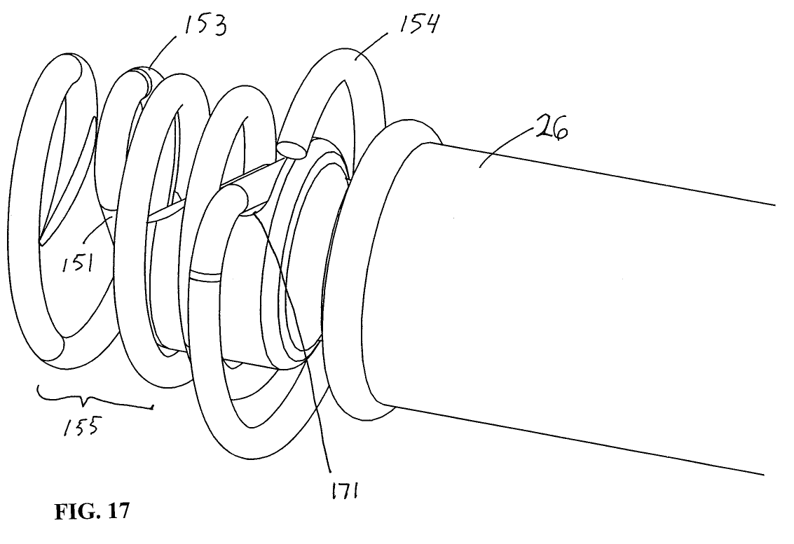

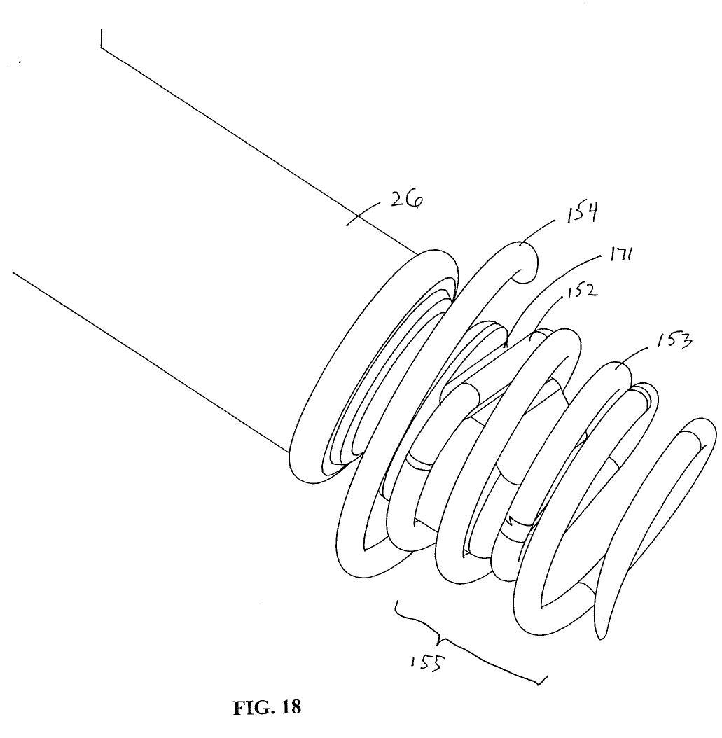

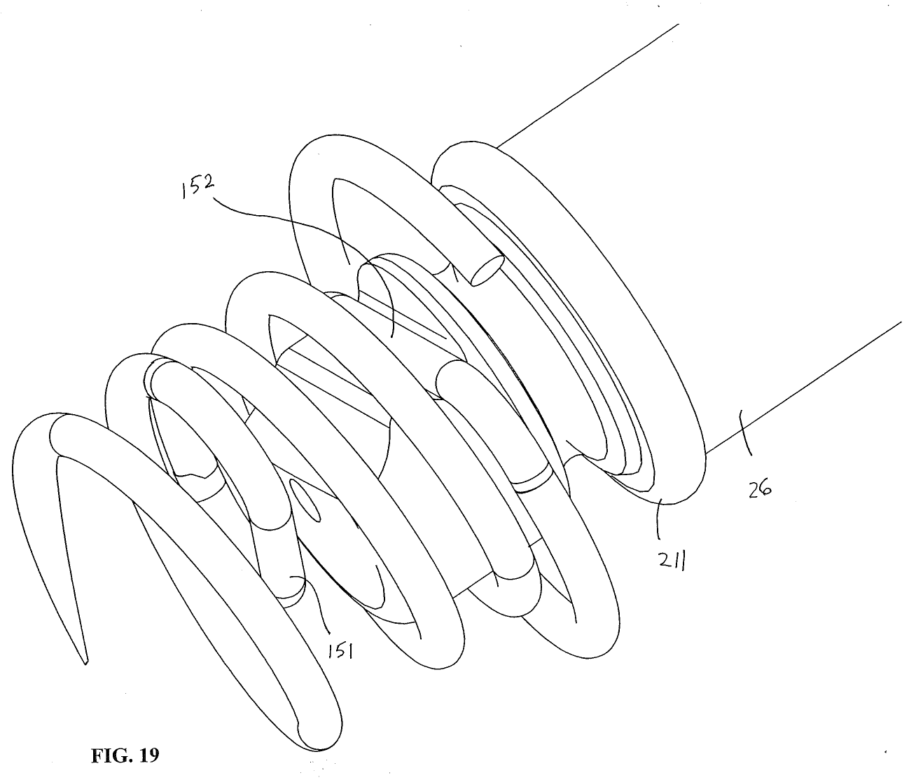

FIGS. 15 and 16 illustrate various views of an embodiment of spiral probe 15. Spiral probe 15 is generally of spiral form. In one embodiment, spiral probe 15 is defined by two or more diameters and multiple different pitches. Additionally, spiral probe 15 includes cross bar 151 adapted to limit the extent to which drive rod 26 can be inserted into the center of spiral probe 15. In this embodiment, cross bar 151 is a generally linear section which spans between opposite sides of spiral probe 15. Spiral probe 15 includes another cross bar 152 which engages a channel portion 171 of driver 26, as shown in FIG. 17. In one embodiment, cross bar 152 is a generally linear section which spans between opposite sides of spiral probe 15.

Spiral probe 15 includes collapsed portion 153, which in this embodiment is approximately 360 degrees, or one turn, from the sharpened end of probe 15. As described in detail hereinafter, collapsed portion 153 limits the extent to which spiral probe 15 enters the fetal tissue field. Spiral probe 15 includes a portion 154 having a greater diameter than a portion 155 proximate to the sharpened end. Enlarged portion 154 engages conductive ring 42 of flex circuit 30 to form a portion of the ECG circuit. As spiral probe 15 is rotated into engagement with the tissue field, portion 154 remains in contact with conductive ring 42 so that regardless of the rotational displacement of spiral probe 15 relative to housing 14, an electrical (ECG) circuit remains intact.

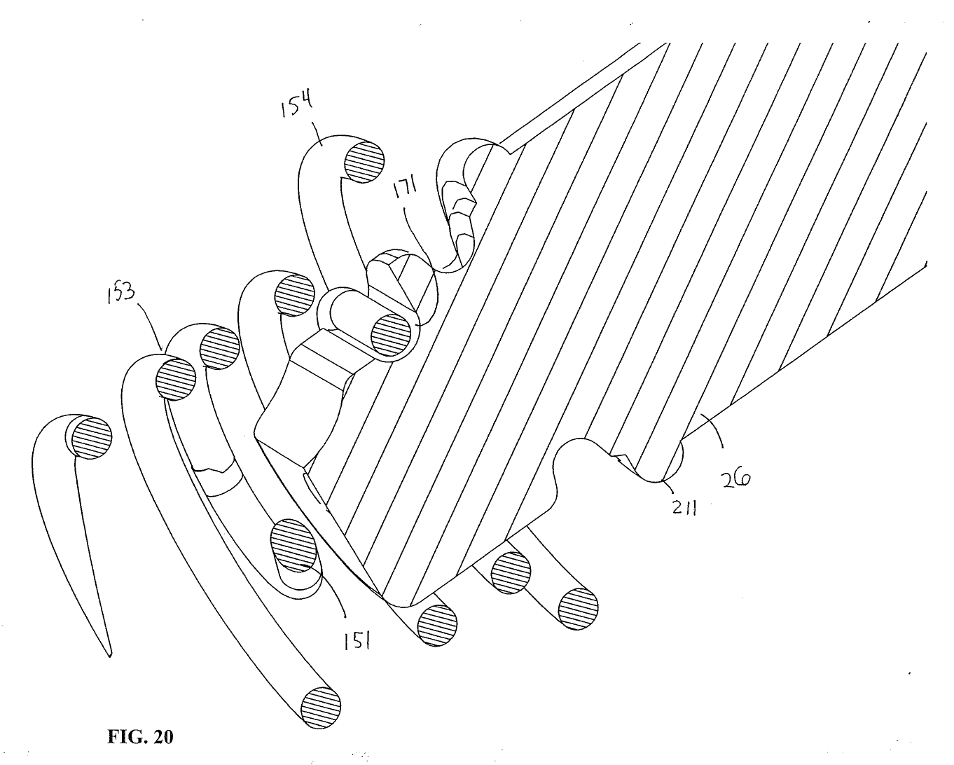

FIGS. 17-20 illustrate in greater detail configured end 27 of drive rod 26 engaged with spiral probe 15 and with other elements of sensor device 10 removed for clarity. These figures depict an engaged configuration of rod 26 and spiral probe 15, such as prior to placement of sensor 10. As shown, cross bar 152 of spiral probe 15 engages channel 171 in drive rod 26.

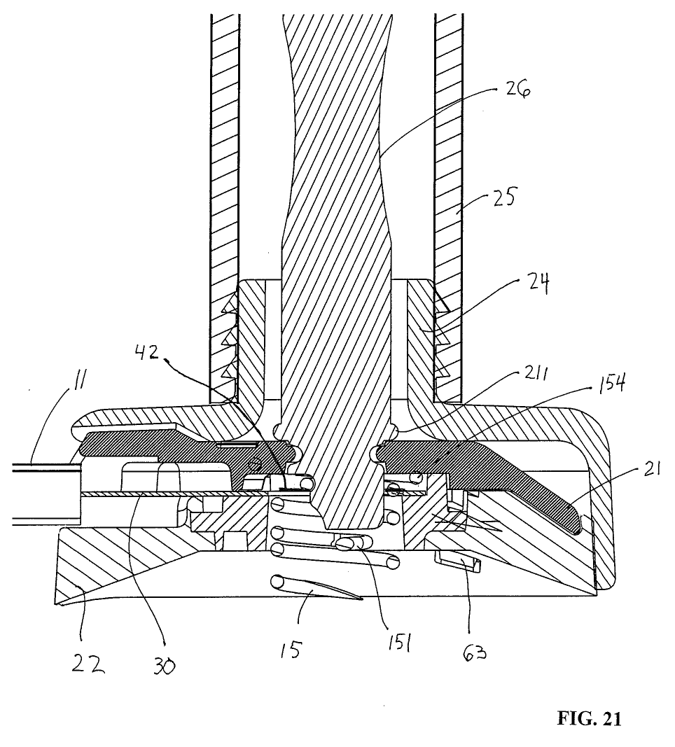

FIG. 21 is a cross sectional view of sensor device 10 prior to placement at the tissue field. Drive rod 26 engages sensor 10 at multiple locations. Shoulder 211 of drive rod 26 engages an upper surface of housing cover 21, limiting the depth to which drive rod 26 can be inserted into sensor 10. Additionally, cross bar 152 engages driver 26 at channel 171 to allow for a torque transfer from driver 26 to spiral probe 15.

During placement of sensor 10 to a tissue field, sensor 10 is introduced through the vagina and attached to the presenting part of the fetus during labor. When sensor 10 is pressed against fetal tissue, the peripheral zone of housing base 22 undergoes elastic deformation into a depressed state. With base 22 in the depressed state, rod 26 is rotated via manipulation of handle 28. With the opposite end of rod 26 engaging spiral probe 15, this rotation of rod 26 results in a 1:1 rotation of spiral probe 15 until axial or rotational disengagement as subsequently described herein. In one embodiment, rotation of rod 26 during sensor 10 placement causes rotation of spiral probe 15 but not housing 14 or lead 11.

As the sharpened end of spiral probe 15 pierces and rotates into the fetal tissue, spiral probe 15 develops a spring force tending to retain housing 14 in place against the fetal tissue. The peripheral zone of base 22 remains engaged on the fetal tissue with surfaces of light emitter 16 and detector 17 in contact or near contact with the tissue field. In one embodiment, the spiral probe is rotated approximately 1 turn into the fetal scalp and the elastic base 22 engages the fetal scalp with an elastic preload.

The unique geometry of spiral probe 15 and rod 26 limits the extent to which spiral probe 15 engages the tissue field. For example, as spiral probe 15 is rotated into the tissue field, the tissue engages the collapsed portion 153 of spiral probe 15. Further rotation causes the tissue field to engage the tip of drive rod 26 and bias the drive rod 26 outwardly and into axial disengagement with spiral probe 15. Drive rod 26 and spiral probe 15 may also be disengaged by application of a pinch force applied to tissue engaging the collapsed portion of spiral probe 15. The pinch force can cause an increased rotational resistance. An increase in rotational resistance can also be exhibited as the spiral probe tip engages denser tissue near the skull. In either case, an increased rotational resistance can result in disengagement of spiral probe 15 from drive rod 26. For example, increased rotational resistance may cause portions of spiral probe 15 to expand radially and release engagement between cross bar 152 and channel 171 of rod 26, at which point rod 26 may rotate without further rotation of spiral probe 15, i.e., the 1:1 rotational relationship between rod 26 and spiral probe 15 is no longer present. Spiral probe 15 and drive rod 26 thus define a detent mechanism whereby upon reaching a predetermined torque, drive rod 26 is released to rotate without further entry of spiral probe 15 into the tissue field.

Upon successful placement of sensor 10 to the tissue field, rod 26 is axially disengaged from sensor 10. In one embodiment, an axial force is applied at the end of rod 26 by compression of retractor 29. Rod 26 and tube 25 can then be withdrawn leaving sensor 10 in place.

In general, the pulse oximetry sensor used in the preferred embodiment of the invention is conventional. Light from the light emitters 16 is directed into the fetal epidermis and reflected back to detector 17. The light transmitted is attenuated by the fetal tissue and then received by detector 17. Processing circuitry associated with the pulse oximetry sensor determines the oxygen saturation of the blood based on the attenuation of the red and infrared light beams. The light beams received by light detector 17 each have a pulsatile and nonpulsatile component. The nonpulsatile components are due to the attenuation of time invariant physiologic blockers such as skin and bone. This is referred to as the DC component. The pulsatile component, on the other hand, represents the attenuation of light during arterial blood flow. This signal is time varying and is often referred to as the AC component. Additionally, the pulsatile components are different for red and infrared light. This difference is due to the fact that hemoglobin and oxyhemoglobin have different optical characteristics. Both hemoglobin and oxyhemoglobin behave similarly with respect to infrared light; however, for red light, the absorption coefficient for hemoglobin is quite different than that of oxyhemoglobin. Thus, the difference in the pulsatile components can be used to derive the level of oxyhemoglobin, and the oxygen saturation of the blood can be computed based on the Lambert-Beers law. In one embodiment, sensor device 10 obtains signals.

The unique aspects of sensor 10, especially spiral probe 15 and its engagement with flex circuit 30 and drive rod 26, fulfill in an excellent manner the objects of a reliable and durable means of attachment to the fetal tissue with acceptable reception of signals for the purpose of measuring vital parameters of a fetus during labor and delivery.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A sensor device comprising:
a housing carrying a light emitter and a light detector adapted for use in a pulse oximetry process of a tissue field; and
a spiral probe carried by the housing and adapted to secure the housing at the tissue field, wherein an elongated probe driver rotates the spiral probe into engagement with the tissue field, with an end portion of the probe driver extending into the spiral probe during a placement procedure.

2. The sensor device of claim 1 wherein the probe driver rotates the spiral probe relative to the housing.

3. The sensor device of claim 2 wherein the probe driver and a portion of the spiral probe define a detent mechanism which limits a torque transferred to the spiral probe during a placement process.

4. The sensor device of claim 3 wherein the detent mechanism includes a cross bar portion of the spiral probe and a channel on the probe driver which engages the cross bar portion.

5. The sensor device of claim 3 wherein the driver and probe are rotated in a 1:1 relationship until a collapsed portion of the spiral probe engages the tissue field, with the probe rotating independently from the spiral probe after engagement between the tissue field and the collapsed portion.

6. The sensor device of claim 5 wherein the spiral probe is defined by two or more different diameters.

7. The sensor device of claim 6 wherein a tissue engaging end of the spiral probe has a smaller diameter than an opposite end, and wherein the opposite end engages a conductor to communication thereto a physiologic signal from the tissue field.

8. The sensor device of claim 7 wherein the opposite end of the spiral probe rotates about a ring-shaped surface of the conductor.

9. The sensor device of claim 8 wherein the conductor is defined upon a surface of a flexible circuit, the circuit coupled to a communications link for communicating the physiologic signal to a remote monitor.

10. The sensor device of claim 8 wherein the light emitter and light detector are mounted on a surface of the flexible circuit.

11. The sensor of claim 1 wherein the end portion of the probe driver is received into the spiral probe to a depth determined by a stop, the stop preventing the end portion from being further extended into the spiral probe.

12. A sensor device comprising:
a housing carrying a light emitter and a light detector utilized in a pulse oximetry process; and
a spiral probe carried by the housing and having a tissue engagement portion and a ring engagement portion, with a diameter of the tissue engagement portion being substantially different than a diameter of the ring engagement portion, wherein the tissue engagement portion engages a tissue field and wherein the ring engagement portion engages a conductor to communicate thereto an electrical signal from the tissue field.

13. The sensor device of claim 12 wherein an end portion of an probe driver is inserted into the spiral probe

14. The sensor device of claim 13 wherein the probe driver and a portion of the spiral probe define a detent mechanism which limits a torque transferred to the spiral probe during a placement process.

15. The sensor device of claim 14 wherein the detent mechanism includes a cross bar portion of the spiral probe and a channel on the driver which engages the cross bar portion.

16. The sensor device of claim 14 wherein the driver and probe are rotated in a 1:1 relationship until a collapsed portion of the spiral probe engages the tissue field, with the probe rotating independently from the spiral probe after engagement between the tissue field and the collapsed portion.

17. The sensor device of claim 12 wherein the ring engagement portion upon a ring-shaped surface of the conductor.

18. The sensor device of claim 17 wherein the conductor is defined upon a surface of a flexible circuit, the circuit coupled to a communications link for communicating the physiologic signal to a remote monitor.

19. The sensor device of claim 18 wherein the light emitter and light detector are mounted on a surface of the flexible circuit.

20. The sensor of claim 13 wherein the end portion of the probe driver is received into the spiral probe to a depth determined by a stop, the stop preventing the end portion from being further extended into the spiral probe.

21. A sensor device comprising:
a housing carrying a light emitter and a light detector utilized in a pulse oximetry process;
a spiral probe carried by the housing; and
a probe driver for rotating the probe, with a distal end of the probe driver being received into the spiral probe to a depth determined by a stop, the stop preventing the driver from being further received into the spiral probe.

22. The sensor device of claim 21 wherein an end portion of the driver extends into the spiral probe.

23. The sensor device of claim 21 wherein the probe driver rotates the spiral probe relative to the housing.

24. The sensor device of claim 21 wherein the probe driver and a portion of the spiral probe define a detent mechanism which limits a torque transferred to the spiral probe during a placement process.

25. The sensor device of claim 24 wherein the detent mechanism includes a cross bar portion of the spiral probe and a channel on the driver which engages the cross bar portion.

26. The sensor device of claim 25 wherein the driver and probe are rotated in a 1:1 relationship until a collapsed portion of the spiral probe engages a tissue field, with the probe rotating independently from the spiral probe after engagement between the tissue field and the collapsed portion.

27. The sensor device of claim 21 wherein the spiral probe is defined by two or more different diameters.

28. The sensor device of claim 27 wherein a tissue engaging end of the spiral probe has a smaller diameter than an opposite end, and wherein the opposite end engages a conductor to communicate thereto a physiologic signal from a tissue field.

29. The sensor device of claim 28 wherein the opposite end of the spiral probe rotates about a ring-shaped surface of the conductor.

30. The sensor device of claim 29 wherein the conductor is defined upon a surface of a flexible circuit, the circuit coupled to a communications link for communicating the physiologic signal to a remote monitor.

31. The sensor device of claim 30 wherein the light emitter and light detector are mounted on a surface of the flexible circuit.

32. A sensor device comprising:
a housing carrying a light emitter and a light detector;
a spiral probe carried by the housing; and
an elongated probe driver for rotating the probe with a 1:1 relationship into engagement with a tissue field, the spiral probe and driver defining a detent mechanism whereby upon reaching a predetermined torque, the driver rotates without further rotation of the probe into the tissue field.

33. The sensor device of claim 32 wherein an end portion of the driver extends into the spiral probe.

34. The sensor device of claim 33 wherein the probe driver and a portion of the spiral probe define a detent mechanism which limits a torque transferred to the spiral probe during a placement process.

35. The sensor device of claim 34 wherein the detent mechanism includes a cross bar portion of the spiral probe and a channel on the driver which engages the cross bar portion.

36. The sensor device of claim 34 wherein the driver and probe are rotated in the 1:1 relationship until a collapsed portion of the spiral probe engages the tissue field, with the probe driver rotating independently from the spiral probe after engagement between the tissue field and the collapsed portion.

37. The sensor device of claim 32 wherein the spiral probe is defined by two or more different diameters.

38. The sensor device of claim 37 wherein a tissue engaging end of the spiral probe has a smaller diameter than an opposite end, and wherein the opposite end engages a conductor to communication thereto a physiologic signal from the tissue field.

39. The sensor device of claim 38 wherein the opposite end of the spiral probe rotates about a ring-shaped surface of the conductor.

40. The sensor device of claim 39 wherein the conductor is defined upon a surface of a flexible circuit, the circuit coupled to a communications link for communicating the physiologic signal to a remote monitor.

41. The sensor device of claim 40 wherein the light emitter and light detector are mounted on a surface of the flexible circuit.

42. The sensor of claim 32 wherein the end portion of the probe driver is received into the spiral probe to a depth determined by a stop, the stop preventing the end portion from being further extended into the spiral probe.

43. A sensor device comprising:
a housing carrying a light emitter and a light detector; and
a spiral probe carried by the housing, the probe being rotatable relative to the housing, with a portion of the probe engaging a tissue field of a patient and another portion of the probe engaging a conductive ring and communicating a physiologic signal from the tissue field.

44. The sensor device of claim 43 wherein an end portion of an probe driver extends into the spiral probe during a placement process.

45. The sensor device of claim 44 wherein the probe driver and a portion of the spiral probe define a detent mechanism which limits a torque transferred to the spiral probe during a placement process.

46. The sensor device of claim 45 wherein the detent mechanism includes a cross bar portion of the spiral probe and a channel on the driver which engages the cross bar portion.

47. The sensor device of claim 44 wherein the driver and probe are rotated in a 1:1 relationship until a collapsed portion of the spiral probe engages the tissue field, with the driver rotating independently from the spiral probe after engagement between the tissue field and the collapsed portion.

48. The sensor device of claim 43 wherein the spiral probe is defined by two or more different diameters.

49. The sensor device of claim 43 wherein a tissue engaging end of the spiral probe has a smaller diameter than an opposite end, and wherein the opposite end engages a conductor to communicate thereto a physiologic signal from the tissue field.

50. The sensor device of claim 49 wherein the opposite end of the spiral probe rotates about a ring-shaped surface of the conductor.

51. The sensor device of claim 50 wherein the conductor is defined upon a surface of a flexible circuit, the circuit coupled to a communications link for communicating a physiologic signal to a remote monitor.

52. The sensor device of claim 51 wherein the light emitter and light detector are mounted on a surface of the flexible circuit.

53. The sensor of claim 51 wherein an end portion of the probe driver is received into the spiral probe to a depth determined by a stop, the stop preventing the end portion from being further inserted into the spiral probe.

54. A sensor device comprising:
a housing carrying a light emitter and a light detector; and
a spiral probe having a sharpened tissue engaging end and an opposite end engaging a conductor for communicating a physiological signal from a tissue field, the spiral probe having a collapsed portion between the tissue engaging end and the opposite end, the spiral probe being rotated to engage a tissue field, with the collapsed portion limiting the depth to which the probe engages the tissue field.

55. A sensor device comprising:
a spiral probe having a tissue engaging end;
a light detector receiving light from a light emitter during an oximetry process; and
a flexible circuit, with the spiral probe being in movable contact with the flexible circuit, and the flexible circuit being coupled to a communications link providing communication of a light detector signal and an probe signal to a remote monitor.

56. The sensor device of claim 55 wherein the light detector and light emitter are mounted on a surface of the flexible circuit.

57. The sensor device of claim 55 wherein the spiral probe rotates upon a surface of the flexible circuit.

58. The sensor device of claim 57 wherein the flexible circuit surface is a ring-shaped conductor.

59. The sensor of claim 58 wherein the spiral probe has at least two different diameters, with a smaller diameter at the tissue engaging end.

60. The sensor of claim 59 wherein the spiral probe has at least two different spiral pitches.

61. The sensor of claim 60 wherein the spiral probe includes a collapsed pitch portion.

62. The sensor of claim 60 wherein the spiral probe defines a stop which limits a depth to which the probe can be rotated into a tissue field.

63. The sensor of claim 60 wherein the spiral probe defines a stop which limits a depth to which an end portion of an probe driver can be inserted into the spiral probe.

64. The sensor device of claim 55 wherein an probe driver and a portion of the spiral probe define a detent mechanism which limits a torque transferred to the spiral probe during a placement process.

65. The sensor device of claim 64 wherein the detent mechanism includes a cross bar portion of the spiral probe and a channel on the driver which engages the cross bar portion.

66. The sensor device of claim 65 wherein the driver and probe are rotated in a 1:1 relationship until a collapsed portion of the spiral probe engages the tissue field, with the driver rotating independently from the spiral probe after engagement between the tissue field and the collapsed portion.

67. A sensor device comprising:
a spiral probe for receiving a physiologic signal from a patient;
a light detector receiving light from a light emitter during an oximetry process of the patient, the light detector being connected to a surface of a flexible circuit; and
a communications link connected to the flexible circuit, the link communicating a signal from the light detector and the physiologic signal from the spiral probe to a remote monitor.

68. The sensor device of claim 67 wherein the spiral probe rotatably engages a conductor defined on a surface of the flexible circuit.

69. The sensor device of claim 68 wherein the spiral probe is defined by multiple different pitches or multiple different diameters or both.

70. The sensor device of claim 67 wherein the spiral probe and an end portion of an probe driver define a detent mechanism which limits a torque transferred to the spiral probe as it engages a tissue field of the patient.

71. The sensor device of claim 67 wherein the spiral probe includes a stop for limiting a depth to which an probe driver end can be inserted into the spiral probe.

72. The sensor device of claim 67 wherein the spiral probe includes a collapsed portion which engages the tissue field during a sensor placement process.

73. A method of using the sensor device of claim 1 comprising:
locating the sensor device proximate to the tissue field;
engaging the tissue field with a sharpened end portion of the spiral probe;
rotating the spiral probe with the probe driver causing the sharpened end to pierce the tissue field;
deforming the spiral probe by further rotation of the probe driver; and
decoupling rotation of the probe driver from the sensor device upon exceeding a predetermined deformation of the spiral probe.

74. The method of claim 73 wherein the deforming causes an increase in a spiral probe diameter.

75. The method of claim 73 wherein the spiral probe is rotated approximately 360 degrees after the tissue field is pierced prior to significant deformation of the probe caused during further rotation.

76. A method of using the sensor device of claim 5, the method resulting in fixation of the sensor device to the tissue field and release of the probe driver from the spiral probe, the method comprising:
locating the sensor device proximate to the tissue field;
engaging the tissue field with a sharpened end portion of the spiral probe; and
rotating the spiral probe with the probe driver until a surface of the tissue field engages the end of the probe driver, wherein further rotation of the probe driver causes the tissue field surface to bias the probe driver away from engagement with the spiral probe, causing the probe driver to be released from the spiral probe.

77. A method of using the sensor device of claim 21 comprising:
locating the sensor device proximate to the tissue field;
engaging the tissue field with a sharpened end of the spiral probe; and
rotating the spiral probe with the probe driver until a surface of the tissue field engages the stop and the end of the probe driver, wherein further rotation of the probe driver causes the tissue field surface to bias the probe driver away from engagement with the stop.

78. A method of using the sensor device of claim 32 comprising:
locating the sensor device proximate to the tissue field;
engaging the tissue field with a sharpened end of the spiral probe;
rotating the spiral probe with the probe driver so as to cause a sharpened end to pierce the tissue field; and
continuing to rotate the probe driver until a predetermined torque level is reached, the torque level causing deformation of the spiral probe and release of coupling between the spiral probe and the driver.

79. The method of claim 78 wherein the predetermined torque level is reached subsequent to contact between a collapsed portion of the spiral probe and the tissue field.

80. The method of claim 78 wherein a diameter of the spiral probe increases causing decoupling between the spiral probe and the driver subsequent to contact between a collapsed portion of the spiral probe and the tissue field.

81. A method of using the sensor device of claim 43 comprising:
locating the sensor device proximate to the tissue field;
engaging the tissue field with a sharpened end of the spiral probe;
rotating the spiral probe with the probe driver until the sharpened end reaches a depth within the tissue field; and
deforming the spiral probe by further rotating the probe driver, the deforming resulting in a rotational decoupling between the driver and the spiral probe.

82. The method of claim 81 wherein the deforming results in an increased diameter of at least a portion of the spiral probe which engages a surface of the driver.

83. A method of using the sensor device of claim 54 comprising:
locating the sensor device proximate to the tissue field;
engaging the tissue field with a sharpened end of the spiral probe; and
rotating the spiral probe with the probe driver until the collapsed portion engages the tissue field, wherein further rotation results in a free spin condition of the probe driver relative to the spiral probe.

84. A system comprising:
a sensor device of claim 1, 12, 21, 32, 43 or 54;
a monitor capable of displaying or recording pulse oximeter information and ECG information; and
a communications link between the sensor device and the monitor, said link communicating information from the sensor device to the monitor for display or recording.

85. The medical system of claim 84 wherein the monitor is remotely located.

86. The medical system of claim 84 wherein the communications link includes a wireless component, a wired component or both.
